(19) United States
(12) Patent Application Publication
(10) Pub. No.: US 2026/0055355 A1
(43) Pub. Date: Feb. 26, 2026

(54) FLEXIBLE BAFFLE FOR USE IN A BIOREACTOR OR MIXER AND METHOD OF INSTALLING BAFFLE

(71) Applicant: GLOBAL LIFE SCIENCES SOLUTIONS USA LLC, Marlborough, MA (US)

(72) Inventors: Nagaraj RAO, Bengaluru (IN); Heather TRACY BEEM, Marlborough, MA (US); Subhanjan CHATTOPADHYAY, Bengaluru (IN); Thomas SMELTZER, Northborough, MA (US); Shrish JOSHI, Bengaluru (IN); Kandakumar MURUGESAN, Bengaluru (IN)

(73) Assignee: GLOBAL LIFE SCIENCES SOLUTIONS USA LLC, Marlborough, MA (US)

(21) Appl. No.: 19/101,956

(22) PCT Filed: Jul. 27, 2023
(86) PCT No.: PCT/US2023/028807

§ 371 Date: Feb. 7, 2025

(30) Foreign Application Priority Data
Aug. 13, 2022 (IN) 202211046265

(51) Int. Cl. C12M 1/00; C12M 3/00

(52) U.S. Cl. CPC C12M 27/20; C12M 23/26; C12M 23/44; C12M 23/46; C12M 23/48

(57) ABSTRACT

A flexible baffle and methods of installation thereof into a bioreactor or mixer vessel are provided. The flexible baffle includes a plurality of segments connected to one another via hinges. Each segment includes at least one alignment element that aids in aligning the segments as the baffle is placed within the bioreactor or mixer vessel, such that the baffle can change from a non-linear shape (e.g., curved) during installation to a linear shape upon completion of installation into the bioreactor or mixer vessel.

### CROSS-REFERENCE TO RELATED APPLICATIONS

This application is a national stage of International Application No. PCT/US2023/028807 filed on Jul. 27, 2023, which claims the benefit of India Application No. 202211046265, filed on Aug. 13, 2022, all of which are hereby incorporated by reference herein in their entireties.

### FIELD

One or more embodiments of the subject matter described herein generally relates to flexible baffles and their use in bioreactors and mixers.

### BACKGROUND

Increasingly, in the biopharmaceutical industry, single use or disposable containers are used. Such containers can be flexible or collapsible plastic vessels or bags that are supported by an outer rigid structure such as a stainless-steel shell or housing. Use of sterilized disposable bags eliminates time-consuming step of cleaning of the housing and reduces the chance of contamination. The bag may be positioned within the rigid housing and filled with the desired fluid for mixing. An agitator assembly disposed within the bag is used to mix the fluid. Existing agitators are either top-driven (having a shaft that extends downwardly into the bag, on which one or more impellers are mounted) or bottom-driven (having an impeller disposed in the bottom of the bag that is driven by a magnetic drive system or motor positioned outside the bag and/or vessel).

Baffles are often placed within the outer rigid structure. Such baffles act as protrusions which press against the container, creating indentations within the containers, when the containers are placed into the rigid structure. These indentations aid in the creation of turbulent flow of fluid within the container when the agitator mixes the fluid, which improves mixing. The indentations also help to prevent vortexing of the fluid when agitation occurs, which can be detrimental to the contents of the container (e.g., cells or cell products). While some bioreactors have baffles integrated directed into the rigid structure, there are many instances where baffles are manually added to the bioreactors, depending upon a user's desires. In larger scale applications (e.g., 500 L and larger), the outer rigid structure (also referred hereto as vessel) is large, and similarly the baffles are tall, heavy, and cumbersome to install.

With reference to FIG. 1, a bioreactor or mixer system 10 of the prior art is illustrated. The bioreactor or mixer system 10 includes a generally rigid bioreactor or mixer vessel 12 mounted atop a base 14 having a plurality of legs 16. The bioreactor or mixer vessel 12 may be formed, for example, from stainless steel, polymers, composites, glass, or other metals, and may be cylindrical in shape, although other shapes may also be utilized without departing from the broader aspects of the invention. A single-use, flexible vessel or bag 15 is disposed within the bioreactor or mixer vessel 12. The bioreactor or mixer vessel 12 can be any shape or size as long as it is capable of supporting a single-use flexible bioreactor bag 15. For example, according to one embodiment of the invention the bioreactor or mixer vessel 2 is capable of accepting and supporting a 10-2000 L flexible or collapsible bioprocess bag 15.

With reference to FIG. 2, which illustrates a top-down view of the bioreactor or mixer vessel 12, a plurality of baffles 14 are located within the bioreactor or mixer vessel 12 around its inner periphery. However, such baffles 14, which are known in the prior art, are either integrated into the bioreactor or mixer vessel 12 or manually added. But when manual addition is to take place, height and size constraints often prevent such installation.

For example, as FIG. 3A illustrates, an XDR2000 bioreactor (e.g., 2000 L bioreactor) offered by Cytiva ®, has a height (H1) of approximately 2600 mm (8.5 ft). The length of a standard baffle 14 necessary for use in a bioreactor of this size is approximately 1500 mm (4.9ft). Inserting such a long baffle 14 into such vessels (e.g., XDR2000) is cumbersome or impossible because of the height of the vessel itself in combination with the ceiling 13 height H2. For example, standard ceiling 13 heights (H2) in the industry are roughly 10 ft to 12 ft, and thus a standard baffle 14 cannot be inserted (e.g., because the distance H2-H1 is too small compared to the length of the baffle 14). Therefor, there is a need for a modified baffle that can be used with bioreactors and mixers where height restrictions prevent the use of standard baffles.

### BRIEF DESCRIPTION

Embodiments of the invention provide a baffle (100, 200) for use with a bioreactor or mixer (10), comprising: a plurality of segments (110, 210), wherein each segment is attached to at least one other segment (110, 210) of the plurality segments by a hinge (120, 220), and wherein each segment includes at least one alignment element (130, 230), the at least one alignment element (130, 230) configured to align each segment (110, 210) with an adjacent segment (110, 210) such that the baffle (100, 200) is configured to move between a non-linear shape and a linear shape.

The baffle includes a top segment (112, 212having a fixing element (113, 213), the fixing element (113, 213) configured to attach to the bioreactor or mixer (10). The baffle also includes a bottom segment with a taper (115, 215).

The at least one alignment element comprises at least one magnet (132, 232), wherein magnets (132, 232) in adjacent segments (110, 210) have opposite polarity such that they attract one another in order to align each segment (110, 210) with the adjacent segment (110, 210).

Alternatively and/or additionally, the at least one alignment element (130, 230) comprises a mechanical element, the mechanical element selected from the group consisting of an alignment rod, a recess, a snap fit element, a tongue, and a groove.

Each hinge (120, 220) of the baffle is attached to two adjacent segments (110, 210), such that adjacent segments (110, 210) can move relative to one another.

The baffle (100, 200) and each of the plurality of segments (110, 210) may have a triangular cross-sectional shape.

When the baffle (100, 200) is in a linear shaped configuration, an outer surface (140, 240) of the baffle (100, 200) has no sharp edges.

Each of the plurality of segments (110, 210) includes a top surface (116, 216) and a bottom surface (118, 218), and wherein the top surface (116, 216) and/or the bottom surface (118, 218) of adjacent segments (110, 210) each include the alignment element (130, 230).

Embodiments of the invention further provide a method of installing a baffle (100, 200) into a bioreactor or mixer (10), comprising: providing the baffle (100, 200) of claim 1; flexing the baffle (100, 200) such that it takes a non-linear shape; inserting a bottom segment (114, 214) of the baffle (100, 200) into a top opening in the bioreactor or mixer (10) and lowering the baffle (100, 200) into the bioreactor or mixer; aligning adjacent segments (110, 210) via respective alignment elements (130, 230) in each of the segments (110, 210), and attaching a top segment (112, 212) of the plurality of segments (110, 210) to the bioreactor or mixer (10).

Aligning adjacent segments (110, 210) comprises magnetically coupling adjacent segments (110, 210), wherein magnetically coupling adjacent segments (110, 210) comprises placing opposing polarity magnets (132, 232) in adjacent segments (110, 210) in proximity to one another.

Alternatively and/or additionally, aligning adjacent segments (110, 210) comprises mechanically coupling adjacent segments, wherein mechanically coupling adjacent segments (110, 210) comprises placing an alignment rod within recess, snapping elements together, or placing a tongue within a groove.

The method further comprises circulating a heat transfer fluid through the baffle (100, 200).

The method further comprises routing at least one tubing or cable through the baffle (100, 200).

The method further comprises attaching at least one bioprocessing accessory to the baffle (100, 200).

Further embodiments provide a baffle (300) for use with a bioreactor or mixer (10), comprising: a unitary structure made from a flexible material such that the baffle (300) is configured to flex between a non-linear shape and a linear shape, wherein the flexible material is rubber.

### BRIEF DESCRIPTION OF THE DRAWINGS

FIG. 1 illustrates a bioreactor according to the prior art.

FIG. 2 illustrates a top-down view of the bioreactor of FIG. 1.

FIG. 3A illustrate difficulties associated with installation of a traditional baffle in the prior art.

FIG. 3B illustrates a benefit of the installation of a flexible baffle, according to embodiments of the invention.

FIGS. 4A-4B illustrate a front and side view, respectively, of a flexible baffle, according to embodiments of the invention.

FIGS. 5A-C illustrate a cross-sectional view, back view, and magnified view of area A of a flexible baffle, according to embodiments of the invention.

FIGS. 6A-6C illustrate a side view, perspective view, and magnified view of area B of a flexible baffle, respectively, of the flexible baffle of FIGS. 4A-5C in a curved state.

FIGS. 7A-7B illustrate a front and side view, respectively, of a flexible baffle, according to further embodiments of the invention.

FIGS. 8A-8B illustrate a cross-sectional view and magnified view of area C of the baffle of FIGS. 7A-7B.

FIGS. 9A-B illustrate a perspective view and magnified view of area C of the flexible baffle of FIGS. 7A-8B.

FIG. 10 illustrates a perspective view of the flexible baffle of FIGS. 7A-8B in a non-linear shape.

FIGS. 11A-C illustrate a perspective view, a side view, and a front view of a flexible baffle according to a further embodiment of the invention.

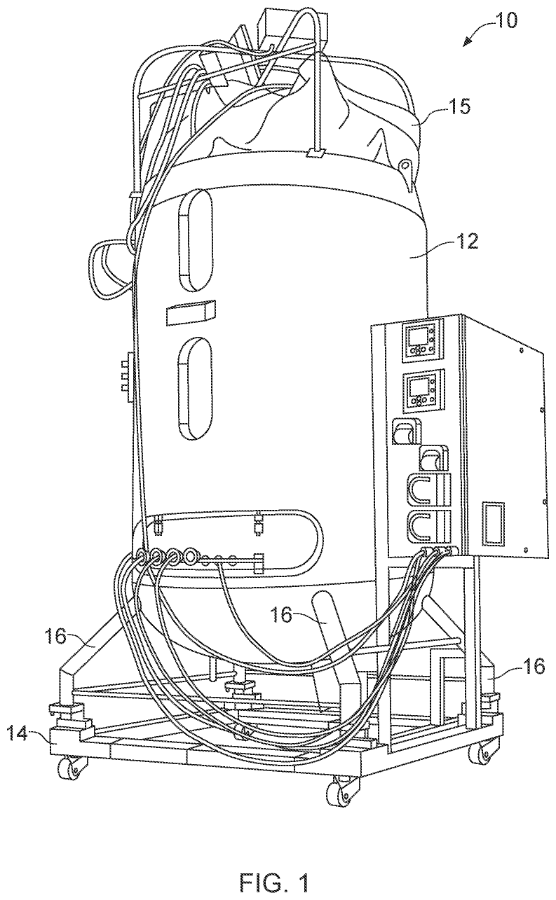

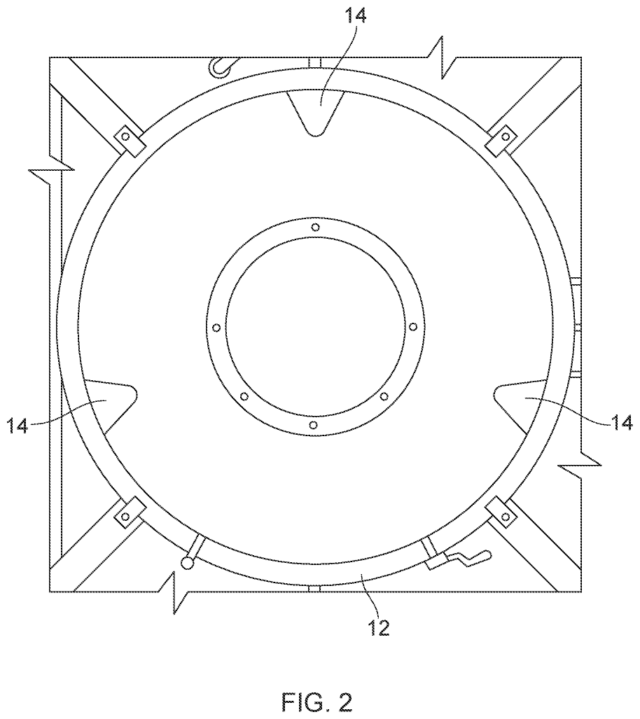

### DETAILED DESCRIPTION

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

It should be noted that the particular arrangement of components (e.g., the number, types, placement, or the like) of the illustrated embodiments may be modified in various alternate embodiments. For example, in various embodiments, different numbers of baffles, as well as the number of segments making up each baffle, can be modified, and is within the scope of the invention.

As used herein, a structure, limitation, or element that is “configured to” perform a task or operation may be particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to”perform the task or operation.

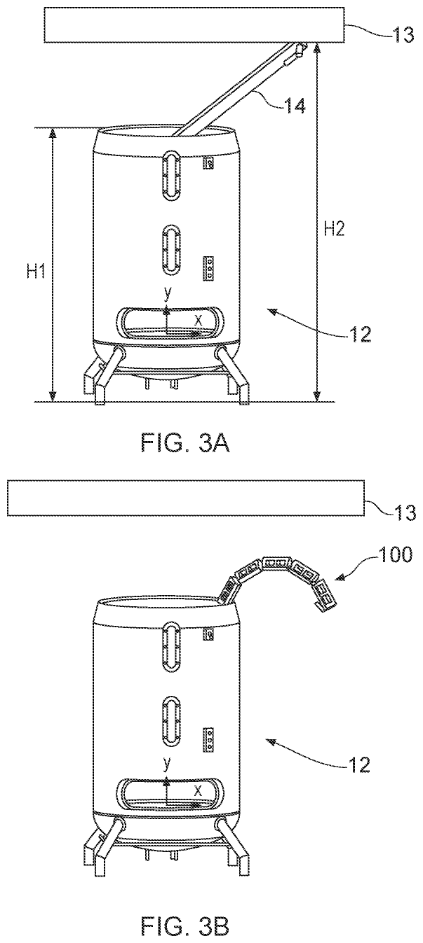

With reference to FIG. 3B, and as will be described in greater detail below, by providing a flexible baffle 100, 200 according to embodiments of the invention, manual addition of baffles to bioreactors or mixers of any size is realized. By providing a flexible baffle 100, 200 with a plurality of hinged segments 110, 120, the baffle 100, 200 can take a non-linear shape (e.g., curved) during installation, such that height restrictions of the installation site are obviated. As the baffle 100, 200 is installed (or once fully placed) within the bioreactor or mixer vessel 12 the plurality of hinged segments 110, 120 align and/or connect with one another via alignment elements 130, 230 such that the baffle 100, 200 takes on a liner shape, and can act to baffle fluids within bag 15, when a fluid within the bag is agitated.

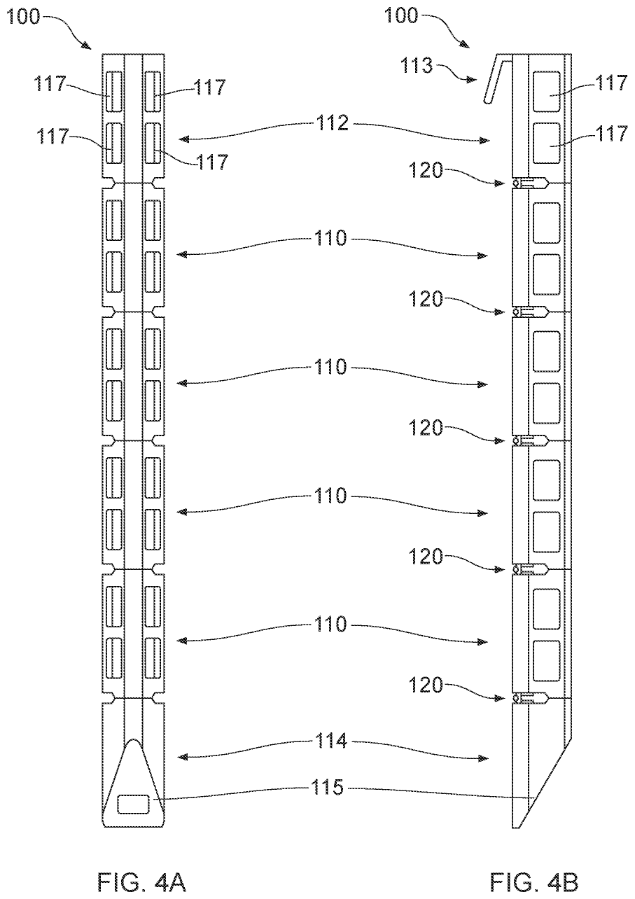

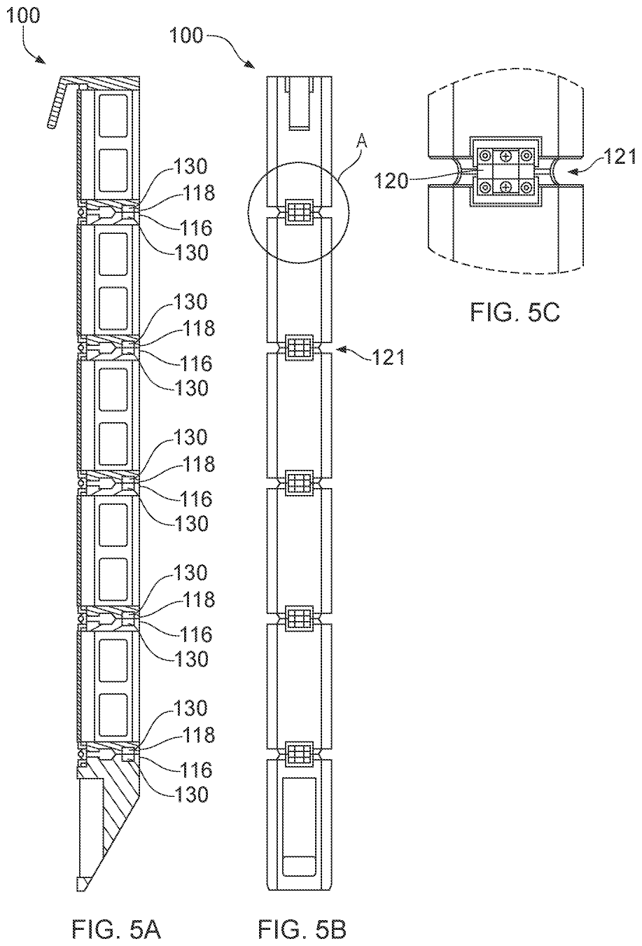

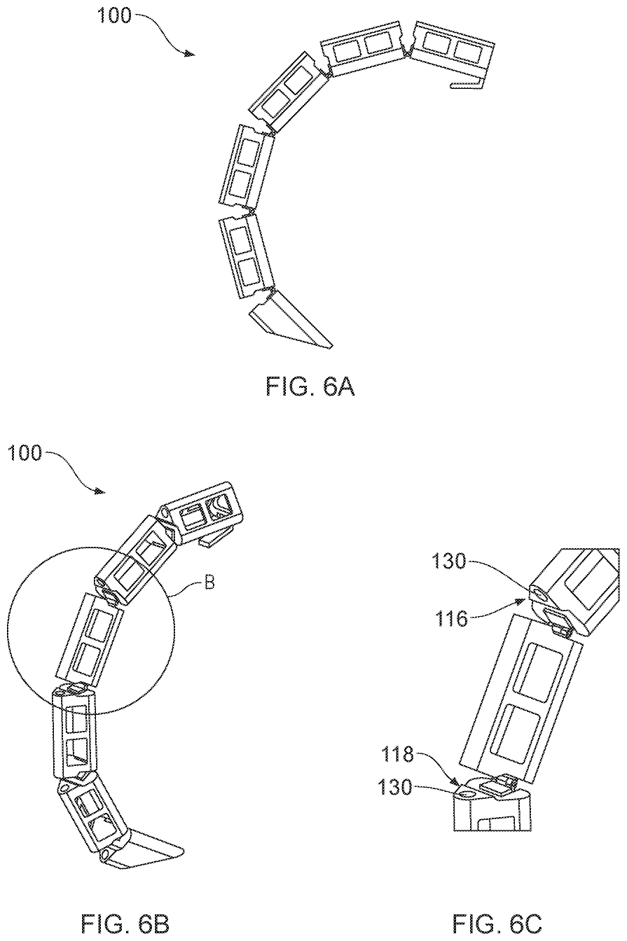

With reference to FIGS. 4A and 4B, a front and side view of a flexible baffle 100, according to embodiments of the invention, are illustrated. The flexible baffle 100 includes a plurality of segments 110. Each segment 110 is attached to at least one other segment 110 of the plurality segments by a hinge 120. As illustrated, top segment 112 is attached to an adjacent segment 110 by a hinge 120 at its lower end, such that the two segments 110 can move relative to one another. A plurality of middle segments 110 are connected to adjacent segments by way of hinges 120 located at the top and bottom ends. Similarly, a bottom segment 114 is attached to an adjacent segment 110 at its top end by a hinge 120. As illustrated by FIGS. 6A and 6B, by constructing the baffle 100 from a plurality of segments 110 connected by hinges the baffle 100 can take a curved or linear shape (see, e.g., FIGS. 4A-5C). The curved shape allows the baffle 100 to be inserted into a bioreactor or mixer vessel 12 with much less clearance (e.g., a distance equal to or less than H2-H1). The baffle 100 can then go from the curved shape into a linear shape by aligning the segments 110 a further described below.

As further illustrated in FIGS. 4 and 4B, the top segment 112 includes a fixing element 113 for attaching the top of the baffle 110 to the bioreactor or mixer vessel 12. The fixing element 113 can be, for example, a flange, a t-bar, or any other mechanical element that can hook onto the top of the bioreactor or mixer vessel 12 wall. The bottom segment 114 includes a tapered surface 115. The tapered surface 115 is implemented in order to help ensure that the baffle 100 does not come into contact with an agitator (e.g., impeller) located within a single-use, flexible vessel or bag 15. Each segment 110 may optionally include a plurality of holes or voids 117 in order to reduce the amount of material necessary for the construction of the baffle 100.

With reference to FIG. 5A, which is a cross-sectional view of baffle 100, each segment 110 may include at least one alignment element 130. Each alignment element 130 is configured to interact or engage with an alignment element 130 in an adjacent segment 110. Each alignment element 130 is located at a bottom surface 118 or top surface 116 of each segment 110 such that abutting top and bottom surfaces 116, 118 each have alignment elements 130. The alignment elements 130 are configured to connect abutting/adjacent segments 110 to one another, such that as (or after) the baffle 100 is installed into the bioreactor or mixer vessel 12, adjacent segments automatically align with each other, thereby aiding in changing the shape of the baffle 100 from non-linear to linear.

In embodiments, each alignment element can 130 be a magnet, an alignment rod, a recess, a snap fit element, a tongue, or a groove. By way of example, an alignment element 130 located at a bottom surface 118 of one segment 110 can be a magnet 132 having a first polarity, while an alignment element 130 located at a top surface 116 of an adjacent, lower segment 110 can be a magnet 132 having a polarity opposite to that of the first polarity. In this way, when the top and bottom surfaces 116, 118 of adjacent segments 110 are brough into proximity with one another the magnets 132 attract, thereby linearly aligning the segments 110 with one another. In other examples, adjacent segments 110 can have correspondingly engaging mechanical elements, such as an alignment rod and groove, snap fit elements, a tongue and groove, etc. In this way, when the top and bottom surfaces 116, 118 of adjacent segments 110 are brought into contact with one another they can be reversibly connected to one another, aiding in ensuring that the baffle 100 maintains a linear shape when installed in the bioreactor or mixer vessel 12.

With reference to FIGS. 5B and 5C, which illustrate a back view and enlarged A view of baffle 100, hinges 120 can be connected to the back and/or underside of each segment 110. Additionally, a gap 121 may exist between adjacent segments on the back side of baffle 100, but not the front side (see, e.g., FIGS. 4A and 4B) in order to accommodate for all or a portion of the hinge 130.

FIGS. 6A-6C provide side, perspective view, and a magnified view of area B, of the flexible baffle 100. As FIGS. 6A and 6B illustrate, baffle 100 can take a non-linear (e.g., curved) shape as well as a linear shape (see, e.g., FIGS. 4A-5B). By taking such a non-linear shape a method of installing the baffle 100 into a bioreactor or mixer vessel 12 can include flexing the baffle 100 such that it takes a non-linear shape; inserting a bottom segment (114, 214) of the baffle (100, 200) into a top opening in the bioreactor or mixer vessel 12 and lowering the baffle 100 into the bioreactor or mixer vessel 12; aligning adjacent segments 110 via respective alignment elements in each of the segments, and, attaching a top segment 112 of the plurality of segments 110 to the bioreactor or mixer vessel 12.

As best shown in FIG. 6C, bottom and top surfaces 118, 116 of adjacent segments 110 include alignment elements 130 that are configured to connect or otherwise mate with one another in order to ensure that baffle 100 maintains a linear shape when installed in bioreactor or mixer vessel 12. This figure also illustrates how the bottom and top surfaces 118, 116 of adjacent segments 110 are not planar. Rather, a front portion of each surface is configured to contact each other, while a back portion of each surface are separated (e.g., by gap 121 shown in FIG. 5C), which accommodates for the hinge 120. Additionally, the hinge 120 can be attached either to the back side of the segments 110 (e.g., as shown in FIGS. 5A-5C) or connected to respective top and bottom surfaces 116, 118 of adjacent segments 110 (e.g., as shown in FIG. 6C). The gap 121, as mentioned above, accommodates for all or a portion of the hinge 130.

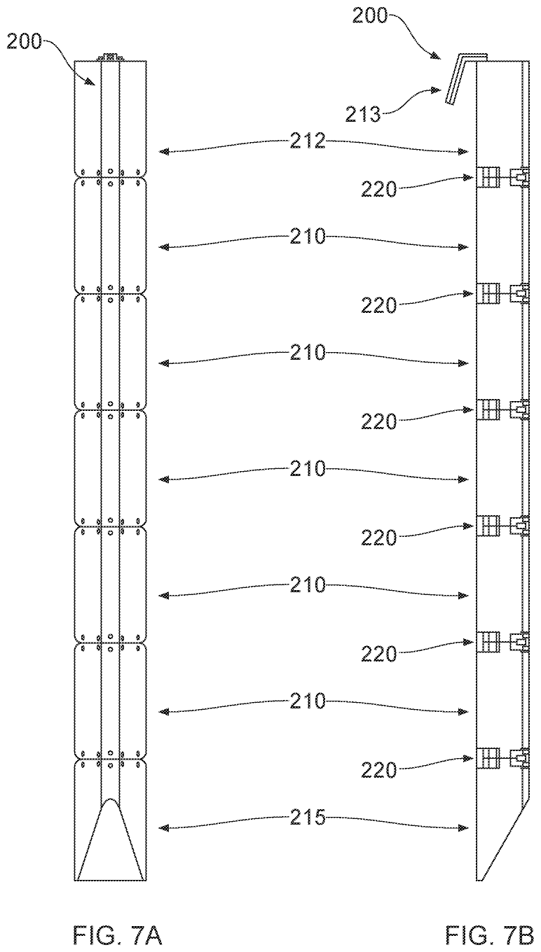

With reference to FIGS. 7A-9B, another embodiment of a flexible baffle 200 is illustrated. Similar to the previous embodiment, flexible baffle 200 includes a plurality of segments 210, where adjacent segments are attached to one another via hinges 220. Each segment 210 includes a top surface 216 and a bottom surface 218. The baffle 200 includes a top segment 212, having a fixing element 213 attached thereto, and a bottom segment 214 having a tapered front surface 215. Alignment elements 230 are located in respective top and bottom surfaces 216, 218 of adjacent segments 210 in order to align the segments 210 when inserting the baffle 200 into a bioreactor or mixer vessel 12. Unlike segments 110, baffle 200 includes segments 210 that do not include any holes. Rather segments 210 may be constructed from a sheet of material, having a completely hollow interior.

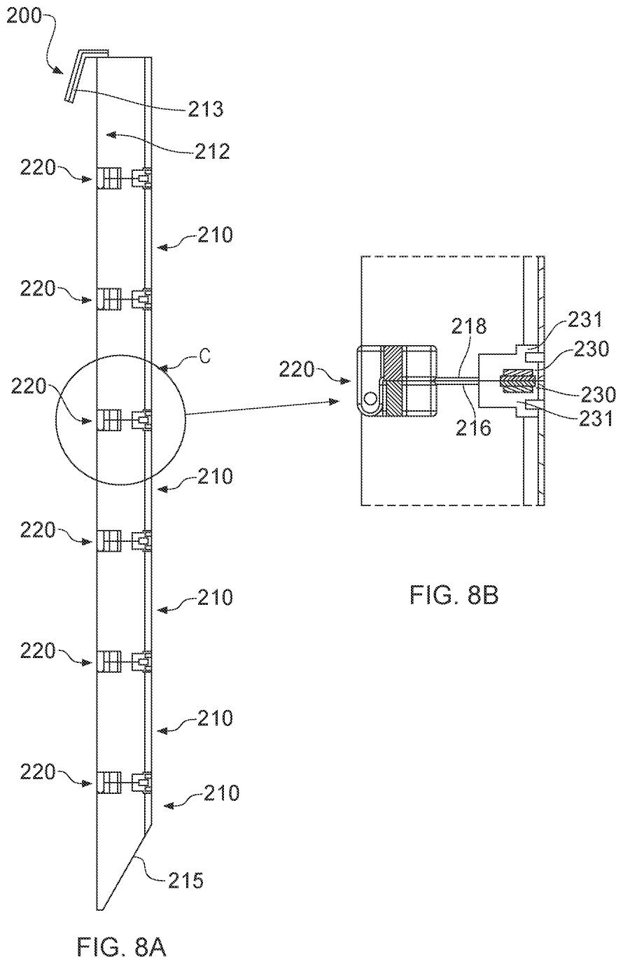

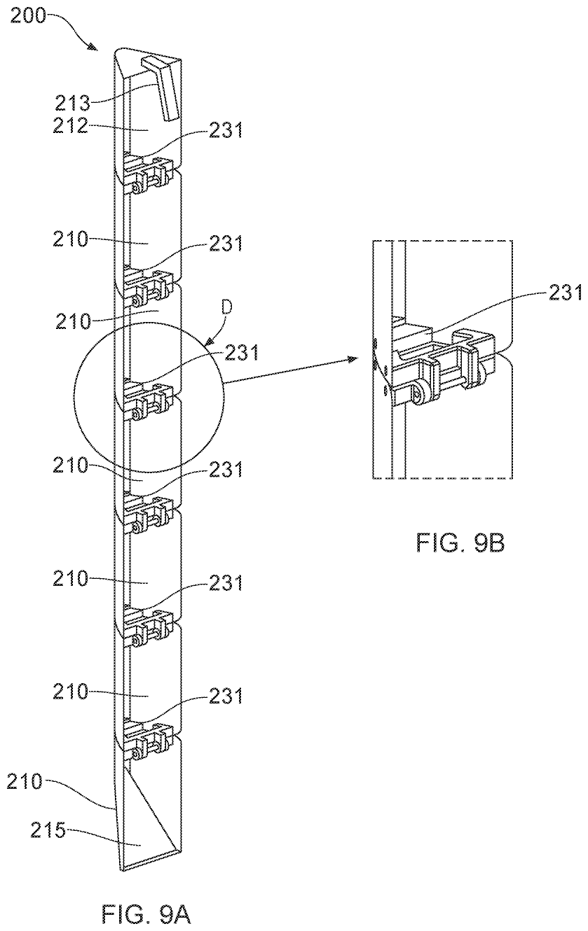

As best shown in FIGS. 8B, 9A and 9B, each alignment element 230 may be located within a housing 231 that is attached to an inner surface of each segment 210 (see, e.g., FIG. 9A). Alternatively, housing 231 may be omitted, and each alignment element can be directly attached (or integral with) a respective segment 210. Like, the previous embodiment, each alignment element can 230 be a magnet 232, an alignment rod, a recess, a snap fit element, a tongue, or a groove. By way of example, an alignment element 230 located at a bottom surface 218 of one segment 210 can be a magnet 232 having a first polarity, while an alignment element 230 located at a top surface 216 of an adjacent, lower segment 210 can be a magnet 232 having a polarity opposite to that of the first polarity. In this way, when the top and bottom surfaces 216, 218 of adjacent segments 110 are brough into proximity with one another the magnets 232 attract, thereby linearly aligning the segments 210 with one another. In other examples, adjacent segments 210 can have correspondingly engaging mechanical elements, such as an alignment rod and groove, snap fit elements, a tongue and groove, etc. In this way, when the top and bottom surfaces 216, 218 of adjacent segments 210 are brought into contact with one another they can be reversibly connected to one another, aiding in ensuring that the baffle 100 maintains a linear shape when installed in the bioreactor or mixer vessel 12.

Additionally, as best shown in FIGS. 9A and 9B, the top and bottom surfaces 216, 218 of adjacent segments 210 may be planar. Unlike the previous embodiment, in the present embodiment the hinge 220 can be accommodated in the empty space on the base side of segments 210.

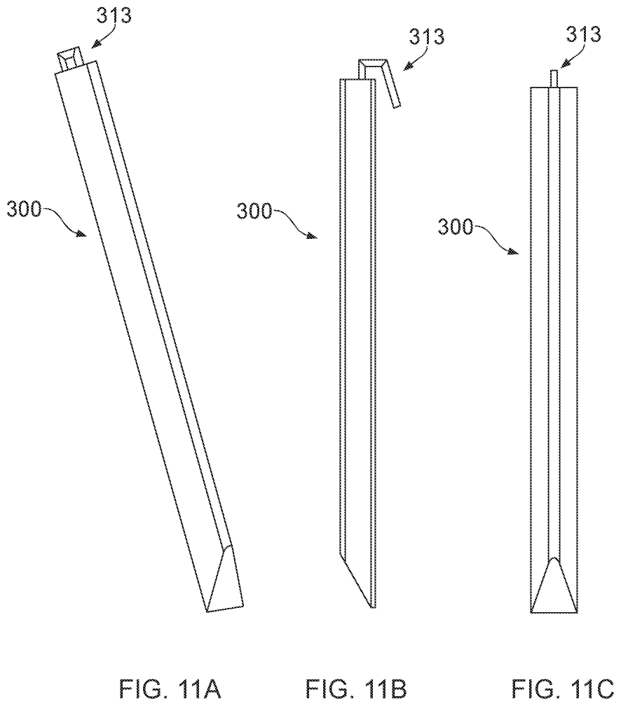

As illustrated in FIGS. 11A-C, and according to further embodiments, a flexible baffle 300 may omit segments and hinges, and instead be of a unitary structure and made from a flexible material. According to embodiments, the flexible material is a flexible polymeric material. According to preferred embodiments, the polymeric material is rubber (e.g., ethylene propylene diene monomer rubber, nitrile butadiene rubber, silicone rubber, etc.). Because the material is flexible the baffle 300 can be bent into a non-linear shape during installation such that baffle 300 takes a curved or semi-curved shape as it is inserted into the bioreactor or mixer vessel 12. As the baffle 300 is loaded into the bioreactor or mixer vessel 12 baffle 300 will flex back into its original linear shape. Similar to the previously described flexible baffles 100, 200, baffle 300 can further include a fixing element 313 for attaching the top of the baffle 300 to the bioreactor or mixer vessel 12. Additionally, a bore or thru hole can be located through the center of baffle 300 along its longitudinal axis such that tubing, wiring, or a temperature regulating fluid can pass therethrough. Compared to the previous embodiments, baffle 300 is cheaper to manufacture, as there are no metal parts, hinges, etc. Additionally, because the baffle may be made from a flexible polymeric material (e.g., rubber) there are no sharp edges or surfaces which could puncture the bag 15. Still further, flexible polymeric material (e.g., rubber) may be selected to be a thermally conductive material such that fluid within the bag 15 can be heated/cooled more efficiently.

Baffles 100, 200, 300 according to embodiments of the invention may have a generally triangular shape when viewing a cross-section in the traverse plane (i.e., top-down view). While a triangular shape is encompassed in a preferred embodiment, other shapes are within the scope of the invention (e.g., semi-circular, rectangular, square, etc.). However, to ensure that the bag 15 is not perforated during installation or use the baffle 100, 200, 300 may include no sharp edges that face into the interior of the bioreactor or mixer vessel 12. For example, as shown in the drawings the front and side edges of the baffle 100, 200, 300 may be curved by means of a chamfer or fillet to prevent the creation of hard/sharp edges that could puncture the bag 15.

The baffles 100, 200, 300 may also be used for additional purposes besides baffling the fluid. For example, the baffles 100, 200 can be utilized as heat transfer surfaces, for tubing and/or cable routing, and/or as a mounting surface for additional accessories. In order to act as a heat transfer element, baffles 100, 200, 30 may have a heat transfer fluid (e.g., gas or liquid) routed through them. For example, because the baffles 100, 200, 30 do not need to be solid a heat transfer fluid can be circulated through the baffles 100, 200, 300, or through tubing located in or behind baffles 100, 200, 300. The temperature of the fluid can be regulated to a desired temperature of the fluid within the bag 15. Since the baffles 100, 200, 300 are in direct contact with the bag 15, thermal energy can be efficiently transferred into or out of the bag 15 as desired. Further, since the baffles 100, 200, 300 are not solid, and a space exists either within or behind the segments 110, 210, tubing or cables necessary for a bioprocessing operation can be routed through the baffles. Still further, because the baffles 100, 200, 300 are fixed structures once installed in the bioreactor or mixer vessel 12, they can act as mounting surfaces for additional accessories, such as filters, cameras, etc.

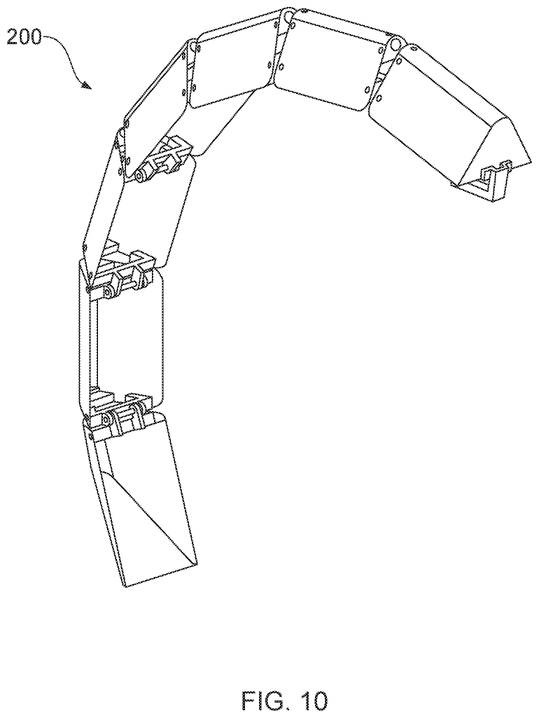

Embodiments of the invention further include methods of installing the aforementioned flexible baffle 100, 200 into a bioreactor or mixer vessel 12. The method may include (i) flexing the baffle 100, 200 such that it takes a non-linear shape, for example, as shown in FIGS. 6A-6C and 10. Once in the non-linear shape, the method may further include (ii) inserting a bottom segment 114, 214 of the baffle 100, 200 into a top opening in the bioreactor or mixer vessel 12 and lowering the baffle 100 into the bioreactor or mixer vessel 12. As the baffle 100, 200 is lowered, segments 110, 210 begin to enter the in the bioreactor or mixer vessel 12. As these segments enter, a further step of the method may include (iii) aligning adjacent segments 110, 210 via respective alignment elements 130, 230 in each of the segments. This step can be accomplished, according to embodiments, by brining magnets 132 of adjacent segments 110, 210 in proximity to one another such that the opposing polarities attract one another. Due to the location of the magnets, adjacent segments will align with one another, creating a linear shape. In other embodiments, mechanical elements (e.g., an alignment rod, a recess, a snap fit element, a tongue, and a groove) are fit together once the adjacent segments 110, 210 are within the bioreactor or mixer vessel 12. For example, (i) an alignment rod of one segment 110 ,210 can be inserted into a recess or hole of an adjacent segment 110 21, (ii) complementary snap fit elements of adjacent segments 110, 210 can be fit together, or (iii) a tongue of a first segment 110, 210 can be fit into a groove of an adjacent segments 110, 210. As a final step, the method can include (iv) attaching a top segment 112 of the plurality of segments 110, 210 to the bioreactor or mixer vessel 12.

According to further embodiments, method of installing a flexible baffle 300 into a bioreactor or mixer vessel 12 are provided. The method may include (i) flexing the baffle 300, such that it takes a non-linear shape. Once in the non-linear shape, the method may further include (ii) inserting the baffle 300 into a top opening in the bioreactor or mixer vessel 12 and lowering the baffle 300 into the bioreactor or mixer vessel 12. As the baffle 300 is lowered, in the bioreactor or mixer vessel 12 the baffle returns to its original linear shape due to the fact that is it made from a flexible polymeric material.

While the above description and embodiments illustrate a flexible baffle having a particular number of segments (i.e., six or seven), flexible baffles 100, 200, 300 according to the invention are not so limited. The baffle 100, 200, 300 can include any number of segments and be of any length (i.e., be sized for use with different bioreactors of different heights). Moreover, while the figures illustrate discrete hinges 120, 220 for connecting adjacent segments 100, 200 it is contemplated that the hinges can be integral parts of the segments themselves (i.e., each segment may have a mechanical element that engages with a complementary mechanical element of an adjacent segment). Moreover, any mechanical elements capable of physically connecting adjacent segments while also allowing them to move relative to one another is within the scope of the invention.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S. C. § 112(f) unless and until such claim limitations expressly use the phrase “means for”followed by a statement of function void of further structure.

This written description uses examples to disclose the various embodiments, and also to enable a person having ordinary skill in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal languages of the claims.

What is claimed is:

1. A baffle for use with a bioreactor or mixer comprising:
a plurality of segments wherein each segment is attached to at least one other segment of the plurality segments by a hinge and
wherein each segment includes at least one alignment element the at least one alignment element configured to align each segment with an adjacent segment such that the baffle is configured to move between a non-linear shape and a linear shape.

2. The baffle of claim 1, wherein a top segment of the plurality of segments includes a fixing element the fixing element configured to attach to the bioreactor or mixer.

3. The baffle of claim 1, wherein a bottom segment of the plurality of segments is has a taper.

4. The baffle of claim 1, wherein the at least one alignment element comprises at least one magnet.

5. The baffle of claim, 4, wherein magnets in adjacent segments have opposite polarity such that they attract one another in order to align each segment with the adjacent segment.

6. The baffle of claim 1, wherein the at least one alignment element comprises a mechanical element, the mechanical element selected from the group consisting of an alignment rod, a recess, a snap fit element, a tongue, and a groove.

7. The baffle of claim 1, wherein each hinge is attached to two adjacent segments such that adjacent segments can move relative to one another.

8. The baffle of claim 1, wherein the baffle and each of the plurality of segments have a triangular cross-sectional shape.

9. The baffle of claim 1, wherein when the baffle is in a linear shaped configuration, an outer surface of the baffle has no sharp edges.

10. The baffle of claim 1, wherein each of the plurality of segments includes a top surface and a bottom surface and
wherein the top surface and/or the bottom surface of adjacent segments each include the alignment element.

11. A method of installing a baffle into a bioreactor or mixer comprising:
providing a baffle;
flexing the baffle such that it takes a non-linear shape;
inserting a bottom segment of the baffle into a top opening in the bioreactor or mixer and lowering the baffle into the bioreactor or mixer;
aligning adjacent segments via respective alignment elements in each of the segments and
attaching a top segment of the plurality of segments to the bioreactor or mixer;
wherein the baffle includes a plurality of segments, each segment being attached to at least one other segment of the plurality segments by a hinge, and each segment including at least one alignment element configured to align each segment with an adjacent segment such that the baffle is configured to move between a non-linear shape and a linear shape.

12. The method of claim 11, wherein aligning adjacent segments comprises magnetically coupling adjacent segments.

13. The method of claim 12, wherein magnetically coupling adjacent segments comprises placing opposing polarity magnets in adjacent segments in proximity to one another.

14. The method of claim 11, wherein aligning adjacent segments comprises mechanically coupling adjacent segments.

15. The method of claim 14, wherein mechanically coupling adjacent segments comprises placing an alignment rod within recess, snapping elements together, or placing a tongue within a groove.

16. The method of claim 11, further comprises circulating a heat transfer fluid through the baffle.

17. The method of claim 11, further comprises routing at least one tubing or cable through the baffle.

18. The method of claim 11, further comprises attaching at least one bioprocessing accessory to the baffle.

19. A baffle for use with a bioreactor or mixer comprising:
a unitary structure made from a flexible material such that the baffle is configured to flex between a non-linear shape and a linear shape.

20. The baffle of claim 19, wherein the flexible material is rubber.
